(19) United States
(12) Patent Application Publication
(10) Pub. No.: US 2016/0228021 A1
(43) Pub. Date: Aug. 11, 2016

(54) ELECTRODE APPARATUS FOR MONITORING ECG

(71) Applicant: LIFURANCE CO., LTD., Daejeon (KR)

(72) Inventors: Jin-Seok LEE, Daejeon (KR); Chang-Won JEONG, Jeollabuk-do (KR)

(21) Appl. No.: 14/733,696

(22) Filed: Jun. 8, 2015

(30) Foreign Application Priority Data
Feb. 6, 2015 (KR) 10-2015-0018455

(51) Int. Cl. A61B 5/0408; A61B 5/00

(52) U.S. Cl. CPC A61B 5/0408; A61B 5/6823; A61B 5/6833; A61B 2562/0209

(57) ABSTRACT

An electrode apparatus for monitoring ECG includes: a sensing area consisting of graphite: a metal member which is attached to the rear surface of the graphite to transmit sensed information; a transmission line which is connected to the metal member and an ECG terminal in order to transmit the sensed information; a support member for supporting the metal member and the graphite; a silicon part of which the rear surface is coated with silicon; and an adhesive patch which has an adhesive material on the inner surface so that the electrode apparatus can be compressed and fixed to a user's skin.

### CROSS REFERENCES

Applicant claims foreign priority under Paris Convention to Korean Patent Application No. 1 0-201 5-001 8455 filed 6 Feb. 2015, with the Korean Intellectual Property Office, where the entire contents are incorporated herein by reference.

### BACKGROUND OF THE INVENTION

1. Field of the Invention

The present invention relates to an electrode apparatus for monitoring a person's ECG which is not affected by environment.

2. Background Art

Heart diseases occur or take a serious turn without previous warning to pose a deadly threat to patients. In order to prevent such heart diseases, technology for monitoring an electrocardiogram (ECG) of a patient, who has a heart disease, in real time has been studied.

The ECG which is one of representative biometric data is a record of action currents generated while heart muscles are expanded and contracted by heart beats, and is recorded through the steps of attaching electrodes to the skin of a human body, measuring action currents according to expansion and contraction of the heart muscles, and describing the measured current data into a graph. In detail, an action potential generated when the heart muscles are expanded and contracted by heart beats causes an electric current transferred to the whole body from the heart, and the electric current generates a potential difference according to parts of the body. The potential difference can be detected and recorded through surface electrodes attached to the skin of the human body. The ECG is used to check the heart for disorder and is also used as the basic measuring method in diagnosis of heart diseases, such as angina, cardiac infarction and arrhythmia.

In general, the electrode induction method used in clinical trials in order to measure electrical abnormality of the heart is to measure biopotential generated while electric excitation generated from the sinoatrial node of the heart is transmitted to the right and left ventricles and the right and left atria, and two or more electrodes are attached to the surface of the human body to measure the biopotential.

Now, the measuring method used the widest is the double-electrode measurement to measure electrical activities of the heart by attaching electrodes to the chest using a flexible band. Such a two-electrode measurement has a problem in that it is difficult to wear the band for a long time because of chest compressions when a user wears the flexible band on the chest. Moreover, the double-electrode measurement has another problem in that the quality of heart beat signals measured is deteriorated if the band is worn on loosely by the user's mistake or humidity of the electrodes is reduced due to a long-term use.

In the meantime, US 2014/0088397A1 (Mar. 24, 2014) discloses fabrication and use of epidermal electrodes which can accurately measure ECG without any influence by external environment by mixing carbon black onto a conductive substrate, which includes electrodes, when a patient on which electrodes are attached is wet with water or is in the water.

The ECG electrode device according to the prior art can measure ECG of the patient who is wet with water, but cannot accurately measure ECG in the water containing sodium chloride, namely, in sea water.

### SUMMARY OF THE INVENTION

Accordingly, the present invention has been made to solve the above-mentioned problems occurring in the prior arts, and it is an object of the present invention to provide an electrode apparatus for monitoring ECG which can accurately measure and monitor a person's ECG in the water, especially in sea water with waves.

To accomplish the above object, according to the present invention, there is provided an electrode apparatus for monitoring ECG including: a sensing area consisting of graphite: a metal member which is attached to the rear surface of the graphite to transmit sensed information; a transmission line which is connected to the metal member and an ECG terminal in order to transmit the sensed information; a support member for supporting the metal member and the graphite; a silicon part of which the rear surface is coated with silicon; and an adhesive patch which has an adhesive material on the inner surface so that the electrode apparatus can be compressed and fixed to a user's skin.

According to the present invention, the electrode apparatus for monitoring ECG can accurately measure ECG without any influence regardless of external environments of a user on which the electrode apparatus is attached, namely, whether the user is in the water, on the ground or in sea water with waves.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a sectional view of an electrode for a living body according to a prior art;

FIG. 2 is a perspective view of an ECG electrode using graphite according to the present invention;

FIG. 3 is an enlarged photograph of graphite according to the present invention and graphite according to the prior art which are enlarged with an electron microscope;

FIG. 4 is a view of a system and a screen for monitoring ECG according to the present invention;

FIG. 5 is a view showing a standard channel 1 ECG electrode which is attached to a patient's body;

FIG. 6 is a view showing procedures for testing performance of the electrode apparatus according to the present invention;

FIG. 7 is a view showing ECG signals measured through the electrode apparatus according to the prior art and the electrode apparatus according to the present invention in general water; and

FIG. 8 is a view showing ECG signals measured through the electrode apparatus according to the prior art and the electrode apparatus according to the present invention in water containing salt.

### DETAILED DESCRIPTION OF THE PREFERRED EMBODIMENT

An electrode apparatus for monitoring ECG according to the present invention includes: a sensing area consisting of graphite: a metal member which is attached to the rear surface of the graphite to transmit sensed information; a transmission line which is connected to the metal member and an ECG terminal in order to transmit the sensed information; a support member for supporting the metal member and the graphite; a silicon part of which the rear surface is coated with silicon; and an adhesive patch which has an adhesive material on the inner surface so that the electrode apparatus can be compressed and fixed to a user's skin.

Hereinafter, reference will be now made in detail to the preferred embodiments of the present invention with reference to the attached drawings.

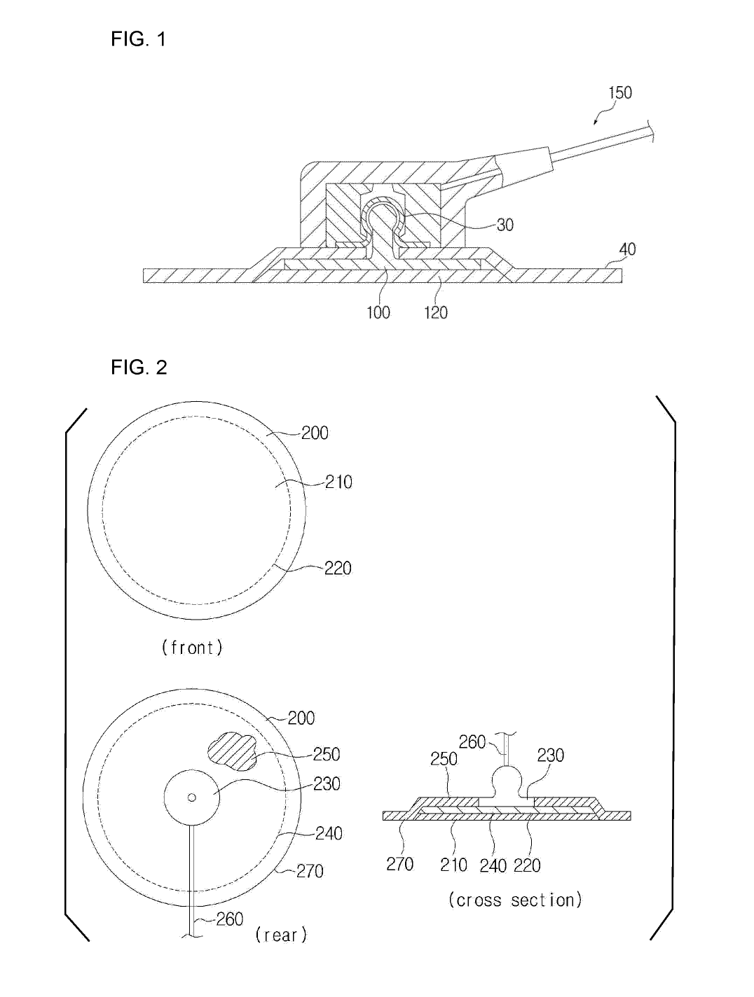

FIG. 1 is a sectional view of an electrode for a living body according to a prior art. A metal electrode 100 which is directly attached to a user's skin to measure ECG, EMG or EEG is made of a conductive material, such as gold, silver, platinum or others, and includes an adhesive material 120 and an adhesive sheet 140 to be perfectly attached to the skin. Furthermore, the metal electrode 100 includes a fixing protrusion 130 which is conductively connected with the metal electrode to transmit information sensed through the metal electrode 100 to a transmission line 150. The electrode for living body according to the prior art depends on external environments, such as the user's skin conditions or moisturized states of the skin, in accuracy.

FIG. 2 is a perspective view of an ECG electrode using graphite according to the present invention. The electrode apparatus 200 according to the present invention uses not metal but graphite 210 as a conductive material on the front surface. The graphite is a mineral belonging to the hexagonal system having the same crystal structure as crystal, is also called ‘black lead’, and is a good conductor of electricity. The electrode apparatus is composed of graphite, and includes a sensing area 220 which is disposed on the front face of the electrode apparatus for sensing bio-signals. The electrode apparatus further includes: a metal member 230 which is attached to the rear surface of the graphite to transmit sensed information; a support member 240 for supporting the metal member and the graphite; a silicon part 250 of which the rear surface is coated with silicon for durability of the electrode apparatus; a transmission line 260 which is connected to the metal member 230 and an ECG terminal in order to transmit information sensed through the metal member 230 to the ECG terminal; and an adhesive patch 270 which has an adhesive material on the inner surface so that the electrode apparatus can be compressed and fixed to the user's skin.

One side and the rear face of the electrode apparatus according to the present invention are coated with an insulation material, such as rubber, epoxy, polyurethane or others.

The graphite in the present invention is processed into a solid form by a predetermined pressure, and the part of the graphite which gets in contact with the user's skin is flat. Moreover, because the graphite is processed into the solid form by the predetermined pressure, any materials other than graphite are not contained.

The graphite of the electrode apparatus according to the present invention is formed in a circle, and is 10 pi to 30 pi in outer diameter and 1 t (1 mm) to 4 t (4 mm) in thickness.

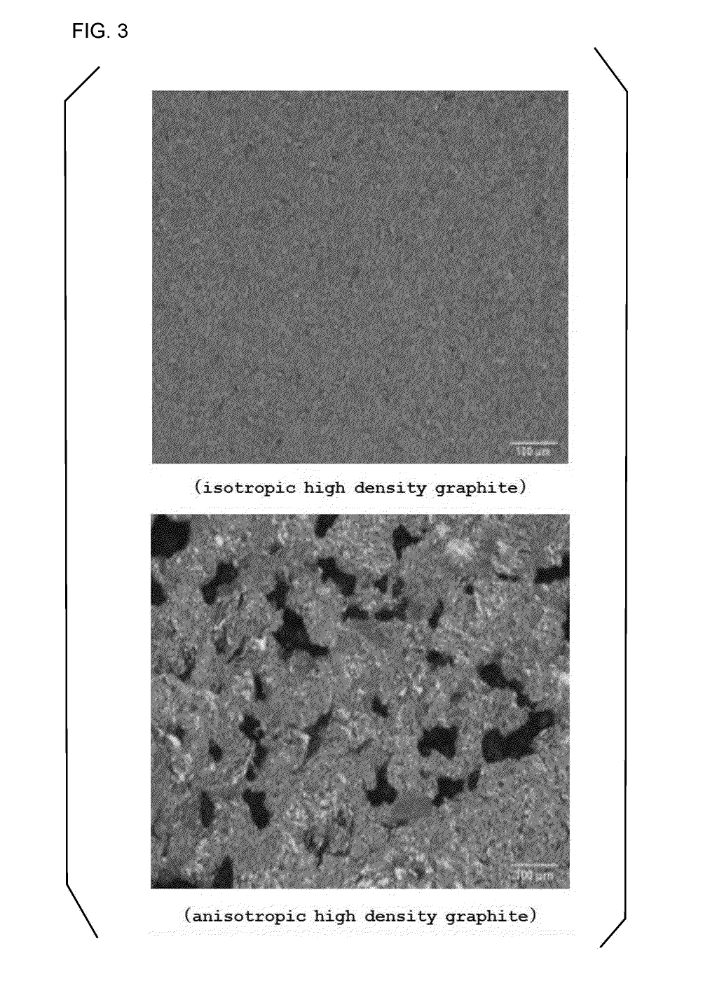

FIG. 3 is an enlarged photograph of graphite according to the present invention and graphite according to the prior art which are enlarged with an electron microscope. The graphite according to the present invention has a minute particle structure, and is made by the hydrostatic pressing method which has the steps of putting raw materials, such as coke or coal-tar pitch, in shielded water with not anisotropic but isotropic high-density, applying pressure of 1 ton per 1 cubic centimeter (cm) equally from every direction, and passing high heat of 3000° C. The graphite with isotropic high density according to the present invention is barely affected by changes in temperature and humidity, compared with the conventional graphite with the anisotropic high density, and is excellent in conductivity.

The graphite used in the electrode apparatus according to the present invention may adopt an anisotropic high density graphite or adopt a material with clay content like a pencil lead.

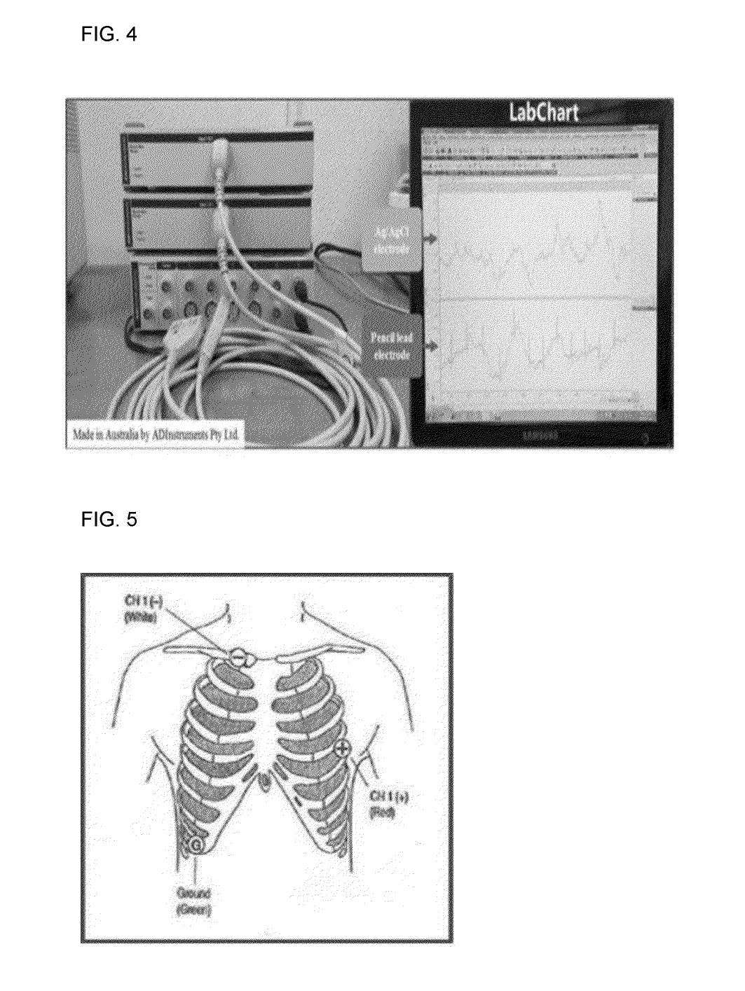

In order to monitor of a person's ECG using the electrode apparatus according to the present invention, a monitoring device illustrated in FIG. 4 is needed. The monitoring device can monitor ECG information from the conventional electrode apparatus and from the electrode apparatus using PowerLab 8/35 and Dual Bio Amp (FE1 35) by LabChart software.

The electrode apparatus according to the present invention measures ECG when a plus electrode and a minus electrode are attached to the heart and the collar bone of the human body and are grounded to one of both sides of the chest. FIG. 5 illustrates the positions of the electrodes.

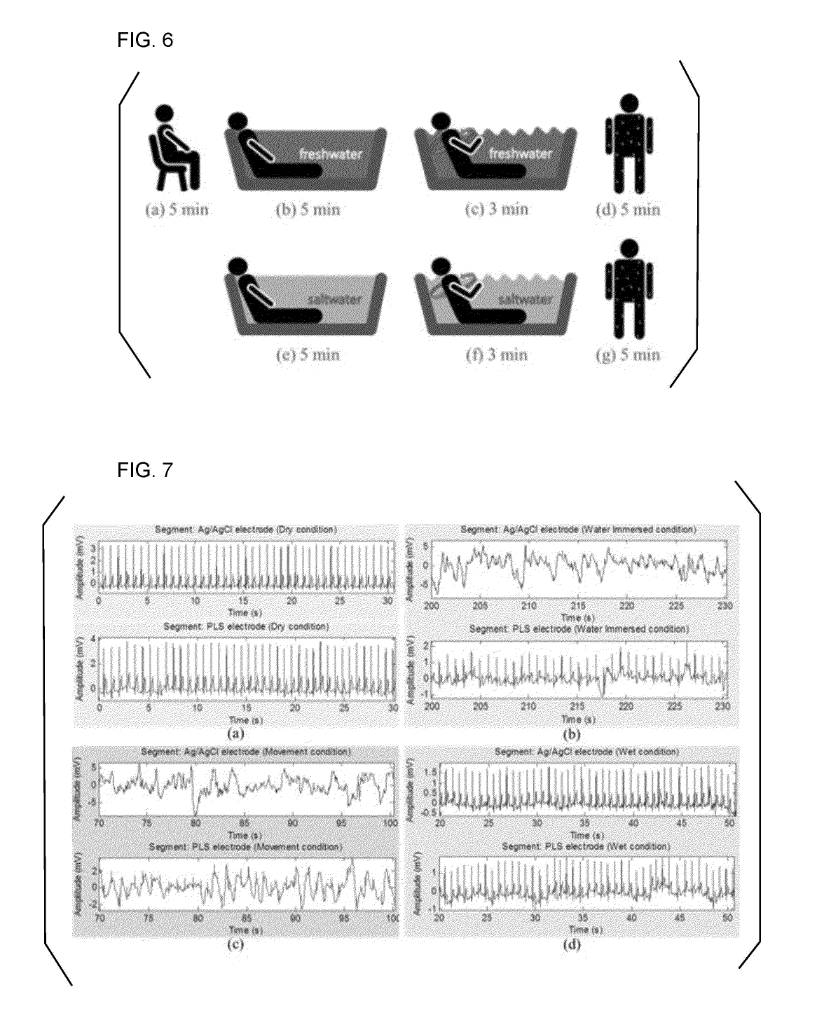

FIG. 6 is a view showing procedures for testing performance of the electrode apparatus according to the present invention. A testee attaches the conventional electrode apparatus onto his or her body and measures ECG while sitting on a chair for five minutes (6a). After that, the testee measures ECG for five minutes after soaking his or her body in water up to the chest (6b), and then, measure ECG for three minutes while twisting and turning (6c). After finishing measurement, the testee measures ECG for five minutes without drying his or her body while standing out of water.

The testee repeats the above-mentioned measurement processes from 6b to 6d after making the same condition as sea water which has salt (6e to 6g).

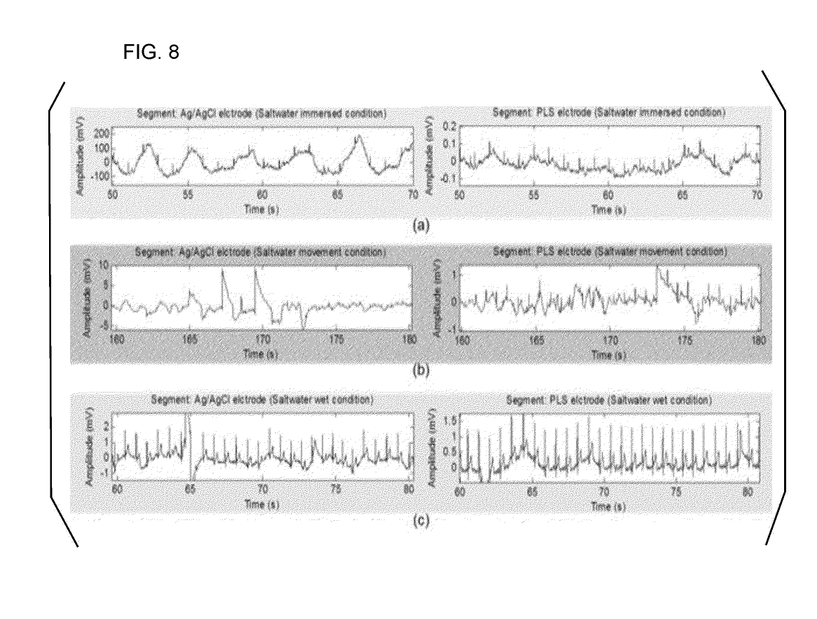

The electrode apparatus automatically stores the testee's ECG information measured through the above test by filtering frequencies of less than 50 Hz through a low-pass filter of LabChart software. FIGS. 7 and 8 show ECG signals obtained through the test of FIG. 6. FIGS. 7a to 7d show ECG information measured in general water. In the general water, the ECG information measured by the conventional electrode apparatus and the ECG information measured by the electrode apparatus according to the present invention are relatively accurate and there is no difference between the ECG information measured by the conventional electrode apparatus and the ECG information measured by the electrode apparatus according to the present invention. However, if the person twisted and turned in the water, FIG. 7c shows less accuracy of the conventional electrode apparatus because noise is caused by waves and it is difficult to accurately measure ECG due to the noise, but the electrode apparatus according to the present invention shows accurate measurement of ECG without any influence by noise phenomenon, such as waves.

FIG. 8 shows that accuracy of the ECG information of the conventional electrode apparatus is so deteriorated in salty water, namely, in sea water, that it is impossible to read the ECG information due to sodium chloride in water. However, the electrode apparatus according to the present invention can accurately extract the ECG information without any influence by sodium chloride in water.

As described above, the electrode apparatus according to the present invention can be widely applied and used to deep-sea divers who work in the deep sea, patients who get rehabilitation treatment in water, skin-scuba divers who enjoy leisure, and other persons because it can accurately extract ECG without any influence by external environments.

It would be understood by those of ordinary skill in the art that various changes in form and details may be made therein without changing the technical idea or essential characteristics of the present invention. Therefore, it would be understood that the above embodiment of the present invention are all exemplified and the present invention is not restricted to the above embodiments. Moreover, it would be also understood that the technical scope of the present invention shall be interpreted by the following claims rather than the detailed description and that all changes or modifications derived from the definition, scope and equivalences of the claims belong to the technical scope of the present invention.

What is claimed is:

1. An electrode apparatus for monitoring ECG comprising:
a sensing area consisting of graphite:
a metal member which is attached to the rear surface of the graphite to transmit sensed information;
a transmission line which is connected to the metal member and an ECG terminal in order to transmit the sensed information;
a support member for supporting the metal member and the graphite;
a silicon part of which the rear surface is coated with silicon; and
an adhesive patch which has an adhesive material on the inner surface so that the electrode apparatus can be compressed and fixed to a user's skin.

2. The electrode apparatus according to claim 1, wherein the graphite is processed into a solid form by a predetermined pressure.

3. The electrode apparatus according to claim 2, wherein the graphite has a minute particle structure with isotropic high density.

4. The electrode apparatus according to claim 2, wherein the graphite has a minute particle structure with anisotropic high density.

5. The electrode apparatus according to claim 3, wherein the part of the graphite which gets in contact with the user's skin is flat.

6. The electrode apparatus according to claim 5, wherein the electrode apparatus is attached to the user's chest and collar bone and is not affected by the user's conditions.

7. The electrode apparatus according to claim 6, wherein the user's conditions includes a dried condition of the user's body, a wet condition of the user's body, a condition that the user is in general water, a condition that the user is in the sea water, a condition that the user moves the body from side to side in the general water, and a condition that the user moves the body from side to side in the sea water.
